(19) United States
(12) Patent Application Publication
(10) Pub. No.: US 2008/0154247 A1
(43) Pub. Date: Jun. 26, 2008

(54) APPARATUS AND METHOD FOR HAIR REMOVAL AND FOLLICLE DEVITALIZATION

(75) Inventors: Joseph L. Dallarosa, Redwood City, CA (US); Leonard C. DeBenedictis, Palo Alto, CA (US); Basil M. Hantash, East Palo Alto, CA (US); Oliver Stumpp, Palo Alto, CA (US)

Correspondence Address: RELIANT / FENWICK;c/o FENWICK & WEST, LLP, 801 CALIFORNIA STREET, MOUNTAIN VIEW, CA, 94041, US

(73) Assignee: Reliant Technologies, Inc., Mountain View, CA (US)

(21) Appl. No.: 11/960,576

(22) Filed: Dec. 19, 2007

Related U.S. Application Data
(60) Provisional application No. 60/871,088, filed on Dec. 20, 2006.

(51) Int. Cl. A61B 18/18

(52) U.S. Cl. 606/3; 606/36; 606/2; 606/9

(57) ABSTRACT

Apparatus and methods for removing hair and/or devitalizing follicles using electromagnetic energy to actively target and treat a hair and/or a follicle are disclosed. The electromagnetic energy can be of a selective or non-selective wavelength. The apparatus comprises a handpiece; a detector that uses electromagnetic energy to detect the presence of a hair and/or follicle in a portion of skin, detect a position of the hair and/or follicle in a plane, and detect an angle at which the hair and/or follicle intersects the plane; a delivery element configured to receive treatment energy from a treatment energy source and deliver the treatment energy to a portion of skin; and a controller that uses feedback from the detector to control the delivery element and/or to control the treatment energy source so as to control the delivery of the treatment energy to the portion of skin via the handpiece in a manner so as to cause hair removal and/or follicular devitalization; wherein the handpiece, the detector, the delivery element and the controller are operably coupled. Optionally, the apparatus can also include an analyzer operably coupled to the handpiece, the detector, the delivery element and/or the controller.

### CROSS-REFERENCE TO RELATED APPLICATION(S)

This application claims priority under 35 U.S.C. 119(e) to U.S. Provisional Patent Application Ser. No. 60/871,088, “Apparatus and Method for Hair Removal and Follicle Devitalization,” filed Dec. 20, 2006. The subject matter of the foregoing is incorporated herein by reference in its entirety.

### FIELD OF THE INVENTION

This invention relates generally to methods and apparatus for hair removal and/or follicle devitalization using electromagnetic energy. More particularly, it relates to methods and apparatus which determine the presence, position and angle of a hair and/or follicle in a region of skin and treat the hair and/or follicle using energy in a manner so as to remove the hair and/or devitalize the follicle.

### BACKGROUND OF THE INVENTION

Numerous methods exist in the art for removing hair and/or for devitalizing hair follicles to eliminate or reduce hair growth. Traditional methods of hair removal include non-permanent methods such as chemical epilation and shaving. Electrolysis is a form of permanent hair removal/follicle devitalization that involves inserting a thin needle into a follicle and delivering electricity into the follicle so as to cause damage to the areas which generate hair. It is a painful, time-consuming and expensive process requiring repeated treatments to produce good results, and has become less popular with the advent of newer treatment methods.

The use of lasers and intense pulsed light (IPL) for hair removal/follicle devitalization has become increasingly popular in recent years. Hair removal/follicular devitalization treatments using lasers and IPL are less painful and more rapid than electrolysis. However, the laser and IPL devices currently marketed are only effective when treating dark colored hairs in light colored skin, as they do not detect and target hairs or follicles but rely on the selective photothermolysis of melanin in the hair.

Thus, there is a need for hair removal/follicle devitalization methods and apparatus which can detect, actively target, and rapidly treat individual hairs and/or follicles using electromagnetic energy.

### SUMMARY OF THE INVENTION

A hair removal and/or follicular devitalization apparatus is disclosed which comprises a handpiece, a detector that detects the presence of a hair and/or follicle in a portion of skin, detects a position of the hair and/or follicle in a plane, and detects an angle at which the hair and/or follicle intersects the plane; a delivery element configured to receive treatment energy from a treatment energy source and deliver the treatment energy to a portion of skin; and a controller that uses feedback from the detector to control the delivery element and/or to control the treatment energy source so as to control the delivery of the treatment energy to the portion of skin via the handpiece in a manner so as to cause hair removal and/or follicular devitalization; wherein the handpiece, the detector, the delivery element, and the controller are operably coupled. In one example, the apparatus comprises a handpiece, a detector, a delivery element and a controller, wherein the handpiece is operably coupled to the delivery element, and wherein the delivery element and the detector are operably coupled to the controller. Optionally, the apparatus can further comprise an analyzer operably coupled to the handpiece, the detector, the delivery element, and/or the controller. Methods of using this hair removal and/or follicular devitalization apparatus in a manner so as to cause hair removal and/or follicular devitalization are also disclosed. The apparatus and methods described herein actively target individual hairs and/or individual follicles, and thus provide fractional treatments using treatment energy at selective and/or non-selective wavelengths.

Other aspects of the invention include methods corresponding to the devices and apparatus described above.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a cross-sectional drawing illustrating a region of skin containing a hair in a follicle.

FIG. 2 is a cross-sectional drawing illustrating skin treated with energy delivered in a fractional manner.

FIG. 3 is a diagram illustrating a hair removal/follicle devitalization apparatus.

FIG. 4 is a top-view drawing illustrating a method of treating a hair and/or follicle using a ring-shaped beam which is centered around the position of the hair and/or follicle.

FIG. 5 is a cross-sectional drawing illustrating treatment depths and offset distances based on the location of a hair and/or follicle.

FIG. 6 is a cross-sectional drawing illustrating how the position and angle of a hair and/or follicle can be used in conjunction with an estimate of the depth of the hair bulge to calculate an offset distance to use in determining a target location for treatment.

FIG. 7 is a perspective drawing illustrating how the position and angle of a hair and/or follicle can be used in conjunction with an offset distance to calculate a target location for treatment.

FIG. 8 is composed of two drawings (8A, a top view and 8B, a cross-sectional view) illustrating a method of treating a hair and/or follicle using a series of target locations aimed along a line based on the position of the hair and/or follicle.

FIG. 9 is a cross-sectional drawing illustrating of a method of treating a hair and/or follicle using a series of target locations aimed along a line based on the position and angle of the hair and/or follicle.

FIG. 10 is a top-view drawing illustrating a method of treating a hair and/or follicle using a series of target locations aimed in a concentric circle based on the position of the hair and/or follicle.

### DETAILED DESCRIPTION OF THE PREFERRED EMBODIMENTS

As used in the specification and the appended claims, the singular forms “a,” “an” and “the” include plural referents unless the context clearly dictates otherwise.

Humans have three different types of hair: lanugo, the fine hair found on fetuses; vellus hair, the short, fine hair that grows in most places on the human body; and terminal hair, the fully developed hair which is generally longer, coarser, thicker, and more highly pigmented than vellus hair. It is terminal hair that is usually the target of hair removal and/or follicular devitalization treatments.

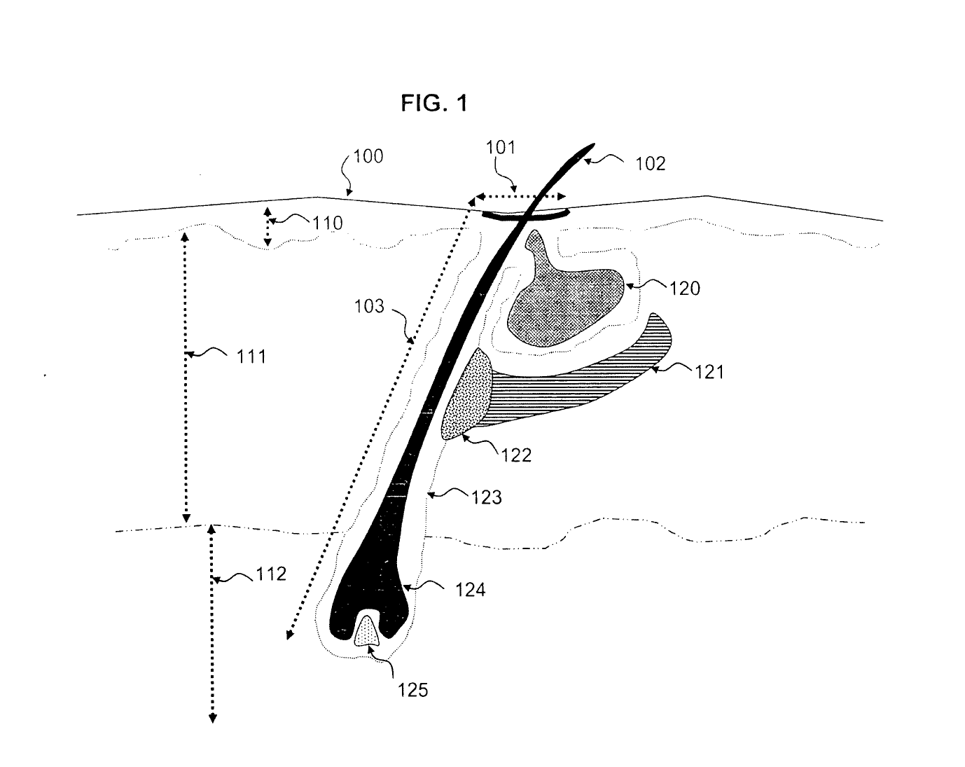

The drawing in FIG. 1 illustrates a region of skin containing a hair (102) lodged in a follicle (103), an involution of the epidermis. The skin surface (100), the opening of the follicle at the surface of the skin (101), and the principle layers of the skin: the epidermis (110), dermis (111), and subcutis (112), are shown.

A sebaceous gland (120), which produces sebum, is shown opening into the follicle (103) near the opening of the follicle at the surface of the skin (101). Below the sebaceous gland (120) and alongside the hair (102) are the arrector pili muscle (121) and the hair bulge (122). The arrector pili (121) is a microscopic band of muscle tissue which connects a follicle to the dermis. The hair bulge (122), at the junction of the arrector pili muscle and the follicle, contains stem cells which create the lower follicle and hair, and which regenerate the hair after it falls out.

While the arrector pili muscle (121) in this cross-section is shown attached to only one side of the hair, it has been demonstrated that, at least for human vellus hair, the arrector pili muscle is present around the entire circumference of the follicle (Narisawa & Kohda (1993) Arrector pili muscles surround human facial vellus hair follicles, Brit J. Dermatol. 129(2): 138-139). It has been suggested that this may also be true for human terminal hairs at least on the scalp (Barcaui et al. (2002) Arrector pili muscle: evidence of proximal attachment variant in terminal follicles of the scalp Brit J. Dermatol. 146(4):657-658). Similarly, in humans, for all types of hair, the hair bulge may also be present around the entire circumference of the follicle.

The root sheath (123) is shown surrounding the hair (102) in the follicle (103). The root sheath can be divided into the external and internal sheaths. The external root sheath is continuous with the stratum basale and stratum spinosum layers of the epidermis. The inner root sheath is adjacent to the hair (102).

At the base of the hair (102), the hair bulb (124) and the hair papilla (125) are shown. The hair is composed mainly of the protein keratin and is formed in the hair bulb. The hair bulb (124) is the lower extremity of the hair that fits like a cap over the hair papilla (125) at the bottom of the follicle (103). The hair papilla is made up of connective tissue and contains the vascular loops which nourish the hair.

Common targets for energy-mediated hair removal/follicular devitalization treatments include the hair, the hair bulge, the root sheath, the hair bulb, the hair papilla, and/or the tissue and vasculature surrounding these structures, as alteration of these structures and/or their surrounding tissue and supporting vasculature may reduce or eliminate hair regrowth. The hair bulge region, including the tissue of the hair bulge and the tissue surrounding the hair bulge is a common target as alteration of the stem cells within and adjacent to the hair bulge may reduce or eliminate hair regrowth. (Orringer et al., (2006) The effects of laser-mediated hair removal on immunohistochemical staining properties of hair follicles, J Am Acad Dermatol 55(3):402-7). The hair bulb region, including the tissue of the hair bulb, the hair papilla, the vasculature surrounding the hair bulb and papilla, and the tissue surrounding the bulb and papilla, is another common target for these treatments, as alteration of the hair bulb and/or the vasculature that provides nutrients to the hair bulb may also reduce or eliminate hair regrowth.

Depending on the treatment parameters used, the clinical effects of treatment on the hair and/or follicle and the surrounding region can include, for example, perifollicular edema and/or erythema; heating, alteration, thinning, shriveling, singeing, charring and/or vaporization of the hair; shedding of the hair from the follicle; heating, alteration, coagulation, necrosis and/or vaporization of the hair matrix, including the follicle, surrounding tissue, and/or supporting vasculature, the hair bulge and/or surrounding tissue, the root sheath and/or surrounding tissue, the hair bulb and/or surrounding tissue, the hair papilla and/or surrounding tissue, the vasculature supplying the hair matrix; and combinations thereof.

The exact mechanisms involved in energy-mediated hair removal/follicular devitalization treatments are not fully understood. Also not fully understood is the correlation between the different clinical effects and effective treatment results (e.g., slowing the rate of hair regrowth without affecting the consistency of the regrown hair; affecting the consistency of the regrown hair so as to make it softer, finer and/or paler; slowing the rate of hair regrowth and affecting the consistency of the regrown hair; permanent inhibition of hair regrowth, and combinations thereof).

Non-selective electromagnetic energy wavelengths are wavelengths that are absorbed more strongly within the skin by chromophores that are homogeneously distributed in the skin, such as, for example, water, than by chromophores that are not homogeneously distributed in the skin, such as, for example, melanin or hemoglobin. Energy sources which emit non-selective energy wavelengths, such as, for example, lasers, are frequently used for skin rejuvenation treatments, but are typically not used for hair removal/follicle devitalization treatments. This is due to the fact that hair removal/follicle devitalization treatments require the treatment energy to be concentrated at target locations deeper within the skin than for skin rejuvenation treatments. When treatment energy is concentrated at target locations deeper within the skin, the use of non-selective wavelengths at levels appropriate for producing effective hair removal/follicle devitalization treatments can cause a high incidence of scarring.

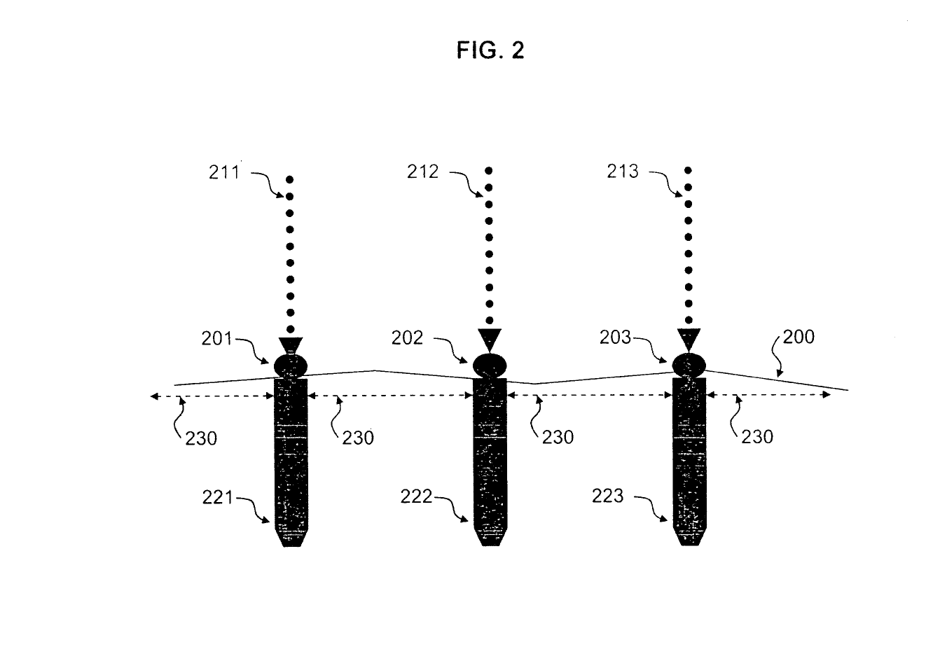

Fractional treatment methods, as illustrated in FIG. 2, can be used with both selective and non-selective energy wavelengths to provide hair removal/follicle devitalization treatments which do not cause a high incidence of side effects such as, for example, scarring. By using fractional treatment methods, within a region of skin (200), only a small number of locations (201, 202, 203) are treated with energy (211, 212, 213), creating a small number of treatment zones (221, 222, 223) where the tissue is exposed to the energy, which are surrounded by untreated zones of tissue (230). This is in contrast with bulk treatment methods, where the entire portion of the tissue is exposed to the treatment energy. The discontinuous treatment zones created by fractional treatment methods result in fewer side effects than do bulk treatments, and allow the untreated tissue to assist in the healing process. Thus, by using the combination of fractional treatment methods and non-selective wavelengths for hair removal/follicular devitalization treatments, higher treatment intensities can be used than can with bulk treatment methods, and highly effective treatments with low levels of side effects can be provided. Additional details about implementations and embodiments of fractional treatment methods can be found, for example, in co-pending U.S. patent application Ser. Nos. 10/888,356 and 60/773,192, which are herein incorporated by reference.

While fractional treatment of skin with energy at non-selective wavelengths can provide effective hair removal and/or follicular devitalization outcomes, the efficiency of the treatment can be further improved by actively detecting hair and/or follicles, determining target locations based on the location of hair and/or follicles, and treating only the target locations. Target locations can include, for example, a hair, a follicle, tissue adjacent to a hair and/or follicle, one or more stem cells within and/or surrounding the bulge region of a hair, tissue within and/or surrounding the bulb region of a hair, one or more blood vessels within and/or surrounding the bulb region and/or papilla region of a hair, and combinations thereof. Determining and treating only the target locations not only minimizes the non-useful energy that is put into the skin, it can reduce the required laser power, speed up the treatment process, reduce the incidence of side effects, and reduce the number of treatments needed to achieve a desired level of hair removal/follicle devitalization. The apparatus and methods described herein detect and actively target hair and/or follicles, and thus provide fractional treatments using energy at selective and/or non-selective wavelengths.

The effectiveness of the outcomes produced by these treatments can be varied by varying the treatment method and parameters used. For example, some treatments can produce permanent damage to a follicle which makes it impossible for a hair to be regenerated by the follicle. Some treatments can alter a follicle such that all future hairs produced by the follicle are of a different, more desirable consistency (e.g., future hairs are softer, finer, and/or paler in color). Some treatments can produce a significant delay in the period of time before a hair is regenerated by a follicle. Some treatments can result in all of a hair or a portion of a hair being destroyed and/or removed from the follicle at the time of treatment, and some treatments can leave a hair in the follicle to fall out later on its own. For the purposes of this application, effective hair removal/follicular devitalization treatment outcomes include: slowing the rate of hair regrowth without affecting the consistency of the regrown hair; affecting the consistency of the regrown hair so as to make it softer, finer and/or paler; slowing the rate of hair regrowth and affecting the consistency of the regrown hair; permanent inhibition of hair regrowth; and combinations thereof.

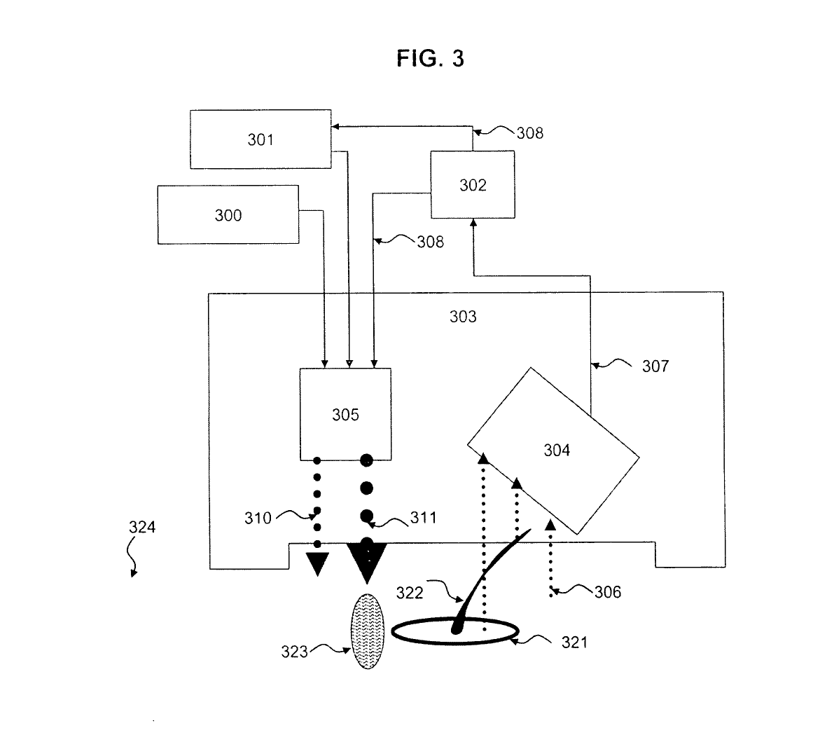

FIG. 3 is a diagram of a hair removal/follicle devitalization apparatus capable of rapidly treating individual hairs and/or follicles by detecting and targeting the hairs and/or follicles in a region of skin. The apparatus consists of a handpiece (303), a detector (304) which may or may not be located in the handpiece, a delivery element (305) which may or may not be located in the handpiece, and a controller (302) which may or may not be located in the handpiece. The handpiece (303), the detector (304), the delivery element (305), and the controller (302) are operably coupled. In one example, the handpiece (303) is operably coupled to the delivery element (305), while the delivery element (305) and the detector (304) are operably coupled to the controller (302). In another example, the apparatus can further comprise a treatment energy source (301) operably coupled to the handpiece, the detector, the delivery element and/or the controller. In another example, the apparatus can further comprise a treatment energy source (301) and a diagnostic energy source (300) operably coupled to the handpiece, the detector, the delivery element and/or the controller. In another example, the apparatus can further comprise an analyzer (not shown in FIG. 3) operably coupled to the handpiece, the detector, the delivery element and/or the controller. In yet another example, the apparatus can include an analyzer operably coupled to the detector and/or the controller. The analyzer can be located in the detector (304) and/or in the controller (302), or can be a separate element.

In addition to the detector (304) and the delivery element (305), the handpiece (303) can optionally further comprise other elements commonly used in such devices which are known to those of skill in the art, such as, for example, a roller which comes in contact with the skin, an offset optical window, a tracking device such as a mouse, etc.

The detector (304) detects the diagnostic effect (306) of the diagnostic energy (310) on the tissue undergoing treatment, and uses the diagnostic effect (306) to distinguish between the skin surface (324) and a hair (322) and/or a follicle (321) and thereby detects the presence of a hair and/or follicle in the portion of skin within the range of the detector. Once the detector (304) detects the presence of a hair and/or follicle in the portion of tissue, it then detects the position of the hair and/or follicle in a plane, and detects the angle at which the hair and/or follicle intersects the plane. The detector can detect the presence, position and angle of a hair and/or follicle by detecting the effect (306) of the diagnostic energy (310) at one point in time, as well as a change in the effect of the diagnostic energy (310) over time, such as, for example, a change in temperature, energy diffraction, energy absorption, energy scattering, capacitance, etc. The plane used by the detector can be the plane of the skin, or it can be a plane created by the diagnostic energy (310). The plane can be a plane in which the diagnostic energy (310) or the diagnostic effect (306) impacts on the hair, the follicle and/or the skin surface.

The detector can detect, for example, temperature, energy diffraction, energy absorption, energy scattering, particular wavelengths of energy, capacitance, etc. In one example, the detector can detect a form of electromagnetic energy. In another example, the detector can be a charge-coupled device, such as, for example, a silicon charge-coupled detector array. In another example, the detector can be a commercially available infrared camera. In yet another example, the detector can be a commercially available near-infrared camera capable of detecting optical energy of wavelengths between about 700 nanometers and about 1000 nanometers.

In one example, the detector can use the effect of the diagnostic energy on the tissue to detect the presence of a particular molecule such as, for example, water, hemoglobin, melanin, myoglobin, lipids, sebum, phytosphingosine, etc, found in or near skin, hair, follicles and/or their surrounding tissue. In another example, the detector can use the effect of the diagnostic energy on the tissue to detect the presence of a particular structure at or below the level of the skin, such as, for example, the opening of a follicle at the surface of the skin, all or a portion of a follicle below the surface of the skin, a sebaceous gland, a hair bulge, a hair bulb, a capillary structure surrounding a follicle, etc. Similarly, the feedback (307) generated by the detector (304) can be of various forms, such as of thermal data, thermal images, infrared data, infrared images, diffraction patterns, absorption spectra, levels of scattering of optical energy, the presence or absence of colors, capacitance data, etc.

The process by which the detector detects the presence, position and/or angle of a hair and/or follicle in a portion of skin can be completely automated. Alternatively, the process by which the detector detects the presence, position and/or angle of a hair and/or follicle can rely in part on input from an operator. In one example, the detector can be comprised of one detector that detects more than one aspect (i.e., presence, position, and/or angle) of the hair and/or follicle. In another example, the detector can be comprised of multiple detectors that detect at least one aspect of the hair and/or follicle.

The delivery element (305) is configured to receive treatment energy from a treatment energy source and deliver the treatment energy to the portion of skin undergoing treatment. The delivery element can include filters and/or optics (e.g., glass, metal, silicon, etc.). In one example, the delivery element can comprise one or multiple delivery fibers. In another example, the delivery element can comprise at least one lens. In another example, the delivery element can comprise a lens array. In another example, the delivery element can comprise one or more rotating wheels containing one or more apperatures or lenses. In another example, the delivery element can comprise an optical scanner and/or a moveable mirror. In another example, the delivery element can comprise a galvanometer scanner (Cambridge Technology, Inc., Cambridge, Mass., USA). In another example, the delivery element can comprise an optical pattern generator using a single rotating component or starburst scanner (see, inter alia, U.S. patent application Ser. No. 11/158,907, which is incorporated by reference).

In one example, the delivery element (305) can be configured to receive and deliver both the treatment energy (311) and the diagnostic energy (310). In another example, a first delivery element can receive and deliver the treatment energy (311), and a second delivery element can receive and deliver the diagnostic energy (310).

The hair removal/follicular devitalization apparatus can optionally include a diagnostic energy source (300) that produces diagnostic energy (310), wherein the diagnostic energy source is operably coupled to the handpiece, the detector, the delivery element and/or the controller. In one example, the diagnostic energy source is operably coupled to the delivery element. The diagnostic energy source can be the same as or different from the treatment energy source. In the example illustrated in FIG. 3, the diagnostic energy source is different than the treatment energy source. The diagnostic energy source can be a source of electromagnetic radiation, including at least one source of ultrasonic energy, radio frequency energy, electricity, ionizing radiation, optical energy, intense pulsed light, infrared light, visible light, ultraviolet light, etc. The diagnostic energy source can include at least one incandescent bulb, laser, light emitting diode, diode laser, and combinations thereof. In one example, the diagnostic energy source is a commercially available light emitting diode.

The diagnostic energy produced by the diagnostic energy source is of sufficient intensity that its effect can be detected by the detector, but is below the intensity needed to produce an effective hair removal/follicle devitalization treatment, and serves only a diagnostic purpose (i.e., is only used for detection purposes). The diagnostic energy can be applied at any angle relative to the plane of the skin, such as, for example, an angle that is substantially perpendicular to the plane of the skin, an angle that is between about 15 degrees and about 85 degrees to the skin, or an angle that is substantially parallel to the plane of the skin (e.g., the beam of diagnostic energy does not impact the surface of the skin but is aimed above the surface of the skin and across the surface of the skin, such that it can impact a hair protruding from the surface of the skin).

The controller (302) uses the feedback (307) from the detector (304) to control the delivery of the treatment energy (311) to a target location in the portion of skin undergoing treatment. The detector feedback (307) used by the controller (302) can include the presence, position, and/or angle of the hair and/or follicle. In one example, the controller can comprise a commercially available computer. In another example, the controller can comprise a laser driver.

In another example, the controller can comprise computer or laser driver circuitry. In yet another example, the controller can convert the detector feedback to control feedback and send the control feedback (308) to at least one of the delivery element (305) and the treatment energy source (301) so as to control the delivery of the treatment energy to the portion of skin undergoing treatment.

The hair removal/follicle devitalization apparatus can optionally include a treatment energy source (301) that produces treatment energy (311), wherein the treatment energy source is operably coupled to the handpiece, the detector, the delivery element and/or the controller. In one example, the treatment energy source is operably coupled to the delivery element. As previously noted, the example illustrated in FIG. 3 has a diagnostic energy source that is different from the treatment energy source. Alternatively, the diagnostic energy can be produced by the same source as the treatment energy. The treatment energy source can be a source of ultrasonic or electromagnetic radiation, including at least one source of radio frequency energy, electricity, ionizing radiation, optical energy, intense pulsed light, infrared light, visible light, ultraviolet light, and combinations thereof. The treatment source of electromagnetic radiation can include at least one incandescent bulb, laser, light emitting diode, and/or diode laser. The treatment energy source can be an aluminum-gallium-arsenide diode laser, an indium phosphide diode laser, an InGaAsP diode laser, a neodymium-doped yttrium aluminum garnet laser, an ytterbium doped fiber laser, an erbium doped fiber laser, a thulium doped fiber laser, and combinations thereof. In one example, the treatment energy source can be a commercially available laser. In another example, the treatment energy source can be a 970 nanometer diode laser (IPG Photonics Corporation, Oxford, Mass., USA).

In one example, the treatment energy can have a wavelength that is selectively absorbed by water, melanin, hemoglobin, myoglobin, lipids, sebum, phytosphingosine, and combinations thereof. In another example, the treatment energy can have a wavelength selected from the group of about 950 nanometers to about 1000 nanometers, about 1064 nanometers, about 1070 nanometers, about 1320 nanometers, about 1520 nanometers to about 1630 nanometers, and combinations thereof. In another example, the treatment energy can have a wavelength of about 970 nanometers.

In another example, the treatment energy can have a wavelength that is selectively absorbed by lipids, such as, for example, wavelengths between about 880 nanometers and about 935 nanometers, between about 1160 nanometers and about 1230 nanometers, between about 1690 nanometers and about 1780 nanometers, between about 2250 nanometers and about 2450 nanometers, and combinations thereof.

In yet another example, the treatment energy can have a wavelength that has a relatively high absorption in lipids and a relatively low absorption in water, such as, for example wavelengths between about 1550 nanometers and about 1850 nanometers, or between about 1690 nanometers and about 1780 nanometers. Selecting wavelengths in these ranges can be particularly advantageous as these wavelengths have relatively low scattering in tissue as compared with shorter wavelengths, and have somewhat lower levels of absorption in tissue as compared to longer wavelengths.

In one example, the treatment energy can have a spot size between about 50 micrometers and about 1000 micrometers at the skin surface. In another example, the treatment energy can have a spot size between about 400 micrometers and about 600 micrometers at the skin surface. In another example, the treatment energy can have pulse energy between about 30 millijoules and about 500 millijoules. In yet another example, the treatment energy can have a pulse energy between about 200 millijoules and about 300 millijoules.

The treatment energy can be sufficient to cause alteration and/or coagulation of the tissue of and/or adjacent to a hair follicle; to cause alteration and/or necrosis of at least one stem cell within and/or surrounding the bulge region of a hair; to cause alteration and/or necrosis of at least one cell within and/or surrounding the bulb region of a hair; to cause alteration and/or coagulation at least one blood vessel within and/or surrounding the bulb region and/or papilla region of a hair; to cause singeing, charring and/or vaporization of a hair; and combinations thereof.

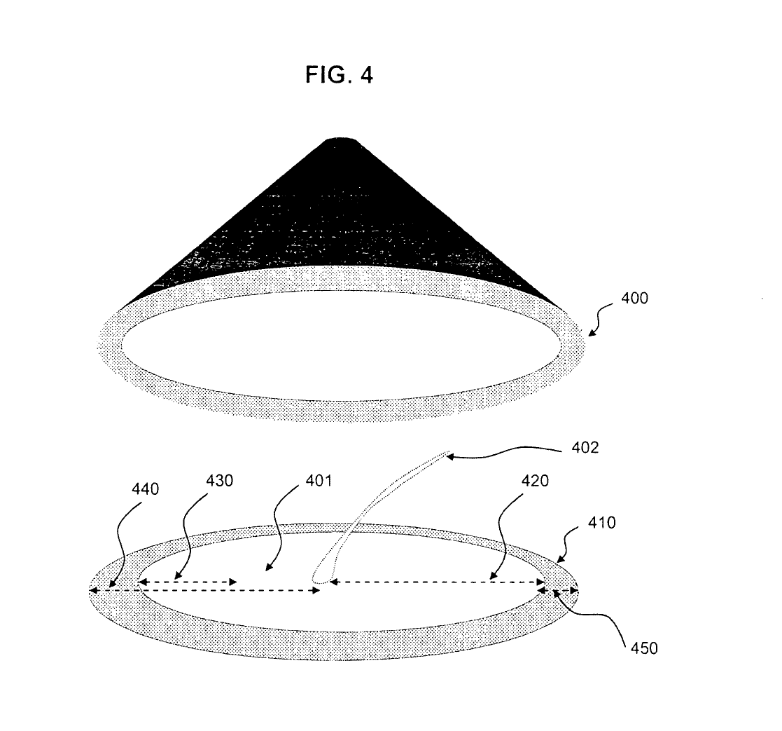

The treatment energy can have a beam in the shape of a circle or an ellipse. Alternatively, the treatment energy can have a beam in the shape of a ring, with the beam centered over the position of the hair and/or follicle. The drawing in FIG. 4 illustrates a beam of treatment energy (400) which has a shape of a ring (410) when it impacts the surface of the skin. The inner (420) and outer (440) radii of the beam can be determined based on the position of a hair (402) (inner radius: 420), a position of a follicle (401) (inner radius: 430), an estimated depth of a hair bulge, and/or an estimated depth of a hair bulb. The width of the beam (450) can be based on a predetermined value such as an estimated width of a hair bulge or hair bulb.

The treatment energy can be applied at any angle relative to the plane of the skin, such as, for example, an angle that is substantially normal to the plane of the skin surface, or an angle between about 15 degrees and about 85 degrees to the skin surface.

In one example, the focus of the beam of treatment energy can be adjusted by the controller. In another example, the pulse energy and/or pulse duration of the beam of treatment energy can be adjusted by the controller. In yet another example, the focus, pulse energy and/or pulse duration can be manually adjusted by the operator. In another example, the angle of the treatment energy as delivered by a delivery element (305, FIG. 3) can be adjusted by the controller. In another example, the timing of the firing of the treatment energy can be adjusted or triggered by the controller. In yet another example, the angle of the treatment energy and/or the timing of the firing of the treatment energy can be controlled manually by the operator.

Optionally, the hair removal/follicle devitalization apparatus can include an analyzer operably coupled to the handpiece, the detector, the delivery element and/or the controller. In one example, the analyzer is operably coupled to the controller and/or the detector. The analyzer can be a separate element, or can be incorporated into another element, such as, for example, the detector or the controller. The analyzer uses feedback from the detector to calculate a target location. The analyzer can calculate a target location by comparing thermal data, diffraction data, absorption spectra, light scatter data, color data, capacitance data, etc.

The analyzer can use additional information, such as a treatment depth and/or an offset distance, to calculate a target location. The treatment depth and/or the offset distance can be based on a predetermined value. For example, the treatment depth and/or the offset distance can be a predetermined value based on one or more factors such as, for example, a region of a body undergoing treatment, a measurement of a depth of a hair bulge, a measurement of a depth a hair bulb, etc. Alternatively, the treatment depth and/or the offset distance can be calculated by the analyzer based on the position, depth, and/or angle of the hair and/or follicle using basic geometry.

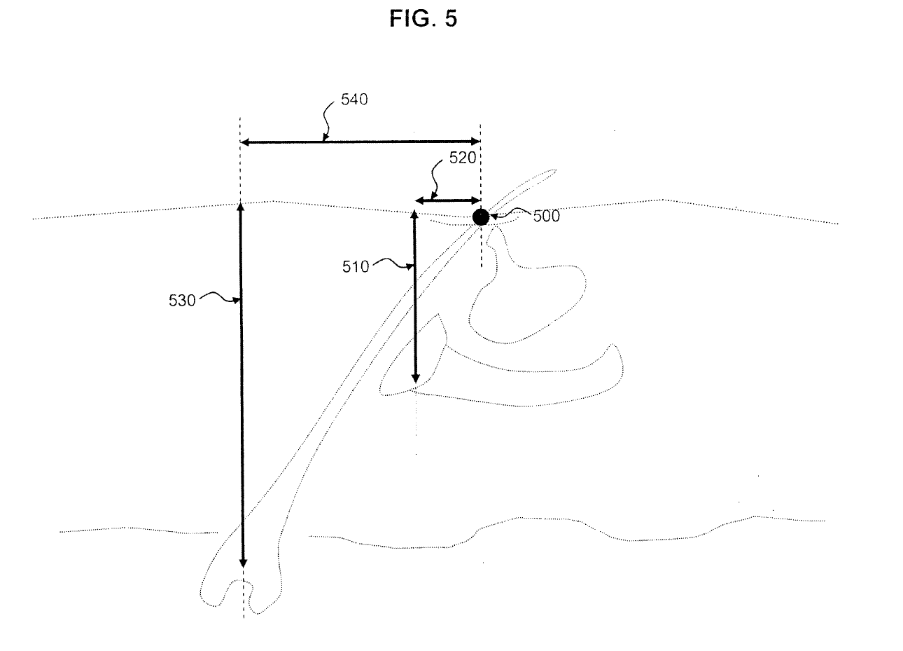

Treatment depth is a vertical depth under the surface of the skin to which the treatment energy must reach so as to treat a desired target beneath the surface of the skin. The offset distance is a horizontal distance as measured on the surface of the skin between a hair and/or an opening of a follicle on the surface of the skin and the position of a desired target beneath the surface of the skin. The drawing in FIG. 5 illustrates two examples of treatment depths and offset distances. Point (500) represents a position of a hair and/or follicle as detected by the detector. Line segment (510) represents a predetermined treatment depth based on an estimated depth of a hair bulge. Line segment (520) represents an offset distance from the position of the hair and/or follicle required for a treatment to be in position to treat the hair bulge under the surface of the skin. Similarly, line segment (530) represents a predetermined treatment depth based on an estimated depth of a hair bulge, and line segment (540) represents an offset distance from the position of the hair and/or follicle required for a treatment to be in position to treat the hair bulb under the surface of the skin.

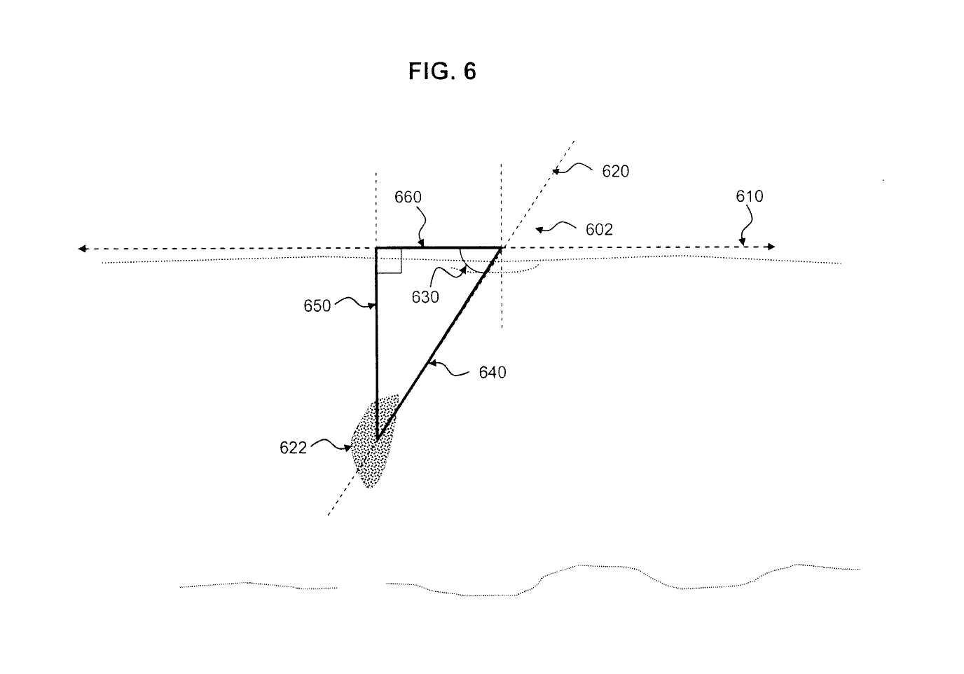

The drawing in FIG. 6 illustrates methods that can be used to calculate treatment depths and/or offset distances based on the position of a hair (602) and/or follicle and the angle (630) at which the hair (602) and/or follicle intersects a plane, where the plane is represented by a line segment (610) substantially parallel to the surface of the skin. Line segment (620) represents a line created based on the position at which a hair (602) intersects the plane (610), and the angle (630) at which the hair and/or follicle intersects the plane (610). Line segment (650) represents the treatment depth required to reach the hair bulge. Line segments (640), (650) and (660) form a right triangle where line segment (640) is the hypotenuse and represents the distance along the hair shaft from the follicle opening to the hair bulge, line segment (650) is the treatment depth, and line segment (660) is the offset distance. Using basic geometry and a combination of predetermined values and measurements for three of the four variables (hair/follicle angle, treatment depth, distance along the hair shaft from the follicle opening to the target, and offset distance), the remaining variable(s) can be calculated using the Pythagorean Theorem as well as the basic properties of triangles. In one example, one or two variables can be estimated and the other variables can be measured or calculated. For example, by rounding the value of the hair/follicle angle to the nearest of a selected set of angles, for example, 30, 45, and 60 degrees, and using the basic relationships between the lengths of the sides of a triangle and estimating the depth of the hair bulge and/or bulb, the distance along the hair shaft from the follicle opening to the target and/or the offset distance can be calculated.

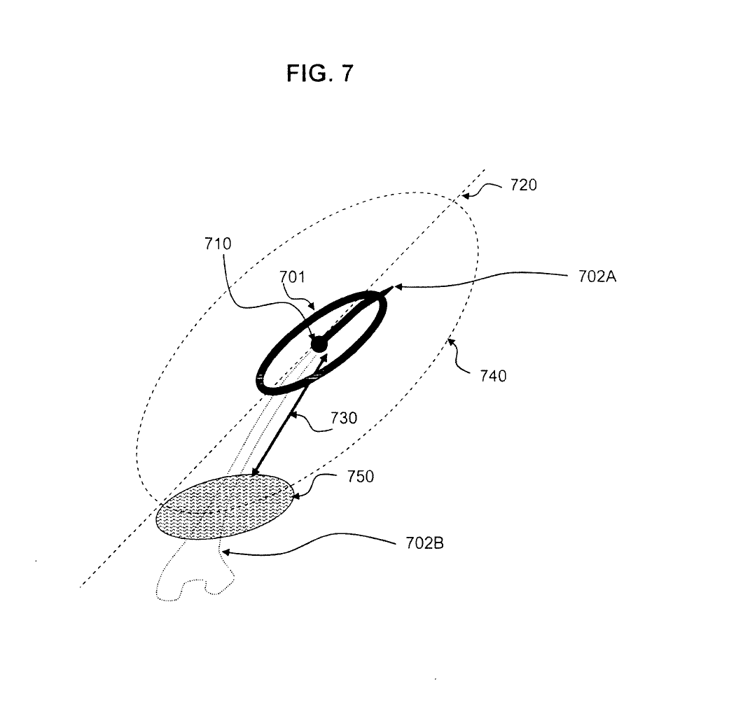

The drawing in FIG. 7 illustrates a perspective view of a method of determining a target location (750) for treatment of a hair (above surface of skin: (702A); below surface of skin: (702B)) in a follicle (opening at surface of skin: 701) using a hair/follicle position (710), a hair/follicle angle (as illustrated by line segment 720) and an offset distance (as illustrated by line segment (730)). The angle at which the hair and follicle slant below the surface of the skin is indicated by the angle at which the hair and/or the follicle intersect the surface of the skin (720), and can be used to estimate the section of a possible treatment circle ((740), radius of offset distance (730)) that should be included in the target location (750) created based on the position of the hair/follicle (710) and the offset distance (730).

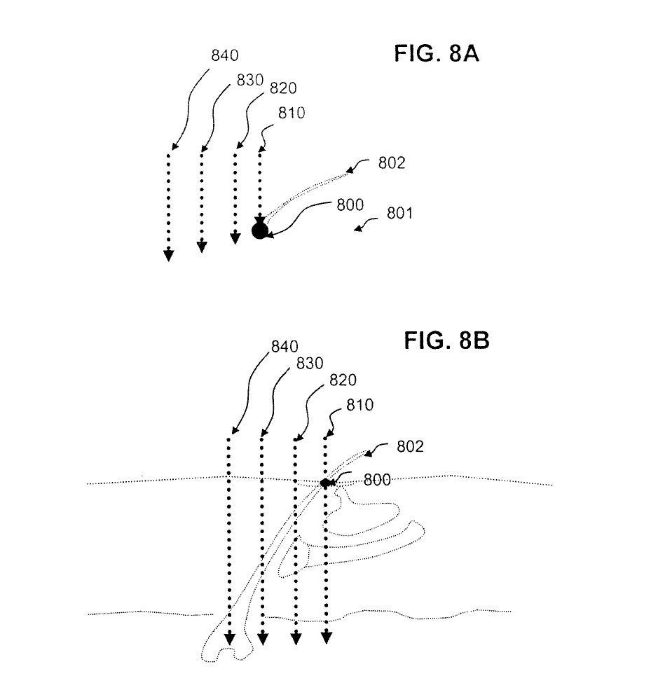

The analyzer can calculate one target location or a series of target locations. The drawings in FIGS. 8A and 8B illustrate top (8A) and cross-sectional (8B) views of a series of target locations (810-840) based on the position (800) of a hair (802) and/or a follicle (801). In this example, the series of treatment locations are along a hair (802), beginning at the detected position (800) of the hair and/or follicle. The number and spacing of the target locations can be predetermined based on the region of the body being treated, an estimate of the depth of the hair bulb, and/or an estimate of the depth of the hair bulge. Alternatively, the number and spacing of the target locations can be calculated by the analyzer. In this example, the treatment depth is the same for all the target locations. In other examples, the treatment depth can increase as the treatment locations get farther away from the position of the hair and/or follicle.

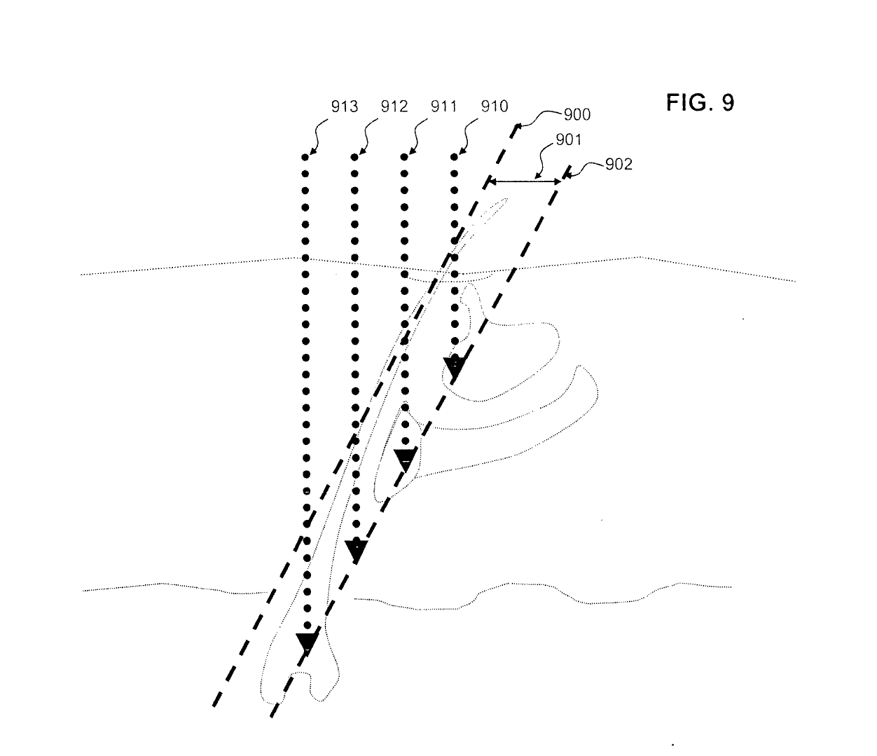

The drawing in FIG. 9 illustrates a cross-sectional view of a series of target locations (910-913) based on the position and angle of a hair and/or follicle. The position and angle of the hair and/or follicle have been used to determine the position of a line segment (900). Optionally, an offset distance (901) can be used to create a second line (902) along which the target locations can be spaced. Line segment (900) represents an estimated path of the follicle below the surface of the skin based on the known position and angle of the hair and/or follicle. Again, the number and spacing of the target locations can be predetermined based on the region of the body being treated, an estimate of the depth of the hair bulb, and/or an estimate of the depth of the hair bulge; or the number and spacing of the target locations can be calculated by the analyzer. The treatment depth can be the same for all the target locations, or can increase as the treatment locations get farther away from the position of the hair and/or follicle, as is illustrated in FIG. 9.

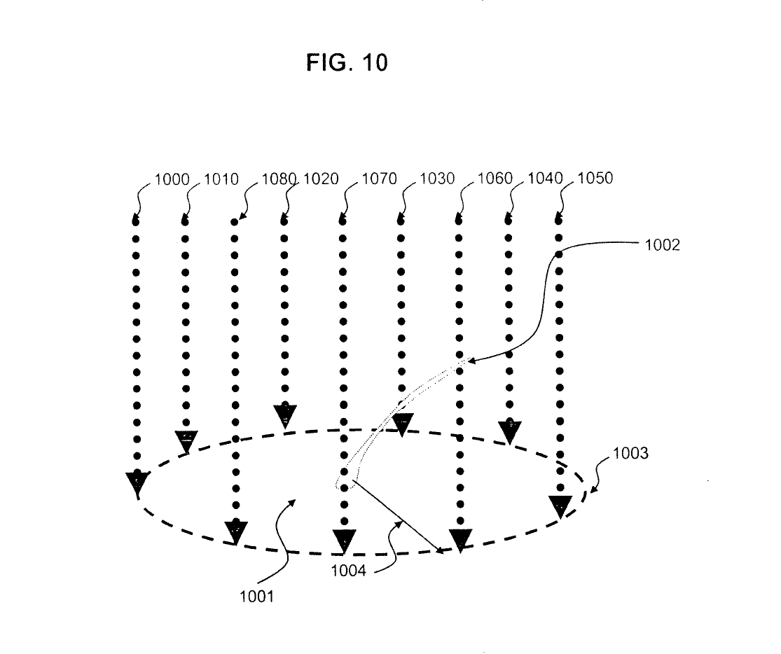

The drawing in FIG. 10 illustrates a series of targets (1000, 1010, 1020, 1030, 1040, 1050, 1060, 1070 and 1080) forming a concentric circle (1003) around the position of a hair (1002) and/or follicle opening at the surface of the skin (1001). The radius of the ring can be determined based on the position of the hair and/or follicle or based on the position of the hair and/or follicle plus an offset distance (radius (1004)).

The method of using the hair removal/follicle devitalization apparatus described herein to treat individual hairs and/or individual follicles in a manner so as to cause hair removal and/or follicular devitalization is also described herein. The method of treatment can use the apparatus described herein to produce particular clinical effects. In one example, the clinical effects can include alteration of the tissue of and/or adjacent to a hair follicle. In another example, the clinical effects can include coagulation of the tissue of and adjacent to a hair follicle. In another example, the clinical effects can include alteration of at least one stem cell within and/or surrounding the bulge region of a hair. In another example, the clinical effects can include necrosis of at least one stem cell within and/or surrounding the bulge region of a hair. In another example, the clinical effects can include alteration of at least one cell within and/or surrounding the bulb region of a hair. In another example, the clinical effects can include necrosis of at least one cell within and/or surrounding the bulb region of a hair. In another example, the clinical effects can include coagulation of at least one blood vessel within and/or surrounding the bulb region and/or papilla region of a hair. In another example, the clinical effects can include alteration of a hair. In another example, the clinical effects can include singeing, charring and/or vaporization of a hair. In yet another example, the clinical effects include a combination of the above effects.

The method of treatment uses the apparatus described herein can be used to produce effective hair removal/follicular devitalization treatment results. In one example, an effective hair removal/follicular devitalization treatment slows the rate of hair regrowth without affecting the consistency of the regrown hair. In another example, an effective hair removal/follicular devitalization treatment affects the consistency of regrown hair so as to make it softer, finer and/or paler. In another example, an effective hair removal/follicular devitalization treatment both slows the rate of hair regrowth and affects the consistency of regrown hair. In another example, an effective hair removal/follicular devitalization treatment results in permanent inhibition of hair regrowth. In yet another example, an effective hair removal/follicular devitalization treatment produces a combination of the above treatment results.

In one example, the method of treatment includes at least one treatment session. In another example, the method of treatment includes more than one treatment session.

### EXAMPLES

### Example 1

A hair removal/follicular devitalization apparatus comprising a handpiece, a detector, a delivery element, and a controller is fabricated and used to remove hairs and/or devitalize follicles. The detector includes an infrared camera with a field of view of the surface of the skin in the treatment region. The detector detects the presence of a hair and/or follicle in a portion of skin, detects a position of the hair and/or follicle in the plane of the surface of the skin, and detects the angle at which the hair and/or follicle intersects the plane of the skin. The apparatus also includes an analyzer. The handpiece includes a mouse to ensure that movement of the handpiece occurs at a rate proportional to hair/follicle detection.

The detector detects the presence and position of the hair and/or follicle by comparing the infrared absorption of the surface of the skin as it moves across the skin. Under illumination by a diagnostic energy source, such as laser energy at a non-selective wavelength between about 1200 nm and about 1400 nm, areas of lower water content, such as hair, absorb less energy than areas of higher water content, such as skin, and this contrast in the levels of absorption is used to determine the presence and position of a hair and/or follicle in a region of skin in a manner that is not dependent upon the color of the hair.

To determine the angle of the hair and/or follicle, a primary diagnostic energy beam is aimed perpendicularly to the surface of the skin, and subsequently refracts off a moveable mirror, such as a galvanometer mirror, before impacting the surface of the skin. The angle at which the beam impacts the surface of the skin can vary, such as, for example, over a range of between about 20 degrees and about 60 degrees. When a beam of larger diameter than a hair is aimed down the shaft of the hair at approximately the same angle as the angle at which the hair protrudes from the surface of the skin, the beam impacts on the surface of the skin in the shape of a symmetrical ring. However, when the beam is aimed down the shaft of a hair at a substantially different angle as the angle at which the hair protrudes from the surface of the skin, the beam impacts on the surface of the skin in the shape of a unsymmetrical ring or a “U” shape, as the shadow of the hair interferes with the shape of the ring formed by the beam. By determining what angle of the beam and/or mirror produces alignment with a particular hair, the detector thus determines the angle of the hair and/or follicle. The detector can be automated so as to detect when the beam and the hair are in alignment, or can rely on operator input to determine when the beam and the hair are in alignment.

The apparatus also includes a display panel which displays a magnified image visible to the operator which is based on the infrared view of the treatment region. During the process of treatment, the operator orients the handpiece in a manner similar to how one would use a hair brush, so that the stroke of the handpiece as it moves across the surface of the skin is in alignment with the direction in which the hairs grow out of the skin. As the handpiece moves across the skin, it detects the presence and position of a hair and/or follicle as described above. Once the detector detects the presence and position of a hair, a magnified view of the hair and/or follicle is displayed on the display panel. The angle at which the hair protrudes from the skin is then determined as described above. The angle of the diagnostic beam is adjusted manually by the operator, or automatically by the apparatus. The operator can manually determine when the angle of the diagnostic beam is alignment with the angle of the hair, or the alignment of the diagnostic beam can be detected automatically by the apparatus.

Once the presence, position and angle of a hair are determined, the apparatus automatically determines a target location and treatment beam is focused onto the target location. Optionally, by viewing the images displayed on the display panel, the operator verifies the target location and then manually fires the treatment beam, and/or verifies a positive treatment outcome by verifying the vaporization, charring or removal of the hair from the follicle following treatment. The treatment beam is produced by an array of 970 nm diode lasers with a combined power of between about 100 watts and about 300 watts, the output of which is grouped into one beam. The spot size of the beam at the point that it impacts the skin is between about 300 micrometers and about 600 micrometers. Other treatment parameters for the treatment energy, such as, for example, irradiance, fluence, pulse energy and pulse duration, are adjusted over a range of parameters so as to achieve a range of effective hair removal and/or follicular devitalization treatment outcomes such as, for example, coagulation of the tissue of and adjacent to a hair follicle, necrosis of stem cells within and surrounding the bulge region of a hair, necrosis of cells within and surrounding the bulb region of a hair, coagulation of blood vessels within and surrounding the bulb region and papilla region of a hair, charring of a hair, vaporization of a hair, and combinations thereof.

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

What is claimed is:

1. A hair removal/follicular devitalization apparatus comprising:
a handpiece;
a detector that
detects the presence of a hair and/or follicle in a portion of skin,
detects a position of the hair and/or follicle in a plane, and
detects an angle at which the hair and/or follicle intersects the plane;
a delivery element configured to receive treatment energy from a treatment energy source and deliver the treatment energy to a portion of skin; and
a controller that uses feedback from the detector to control the delivery element and/or to control the treatment energy source so as to control the delivery of the treatment energy to the portion of skin via the handpiece in a manner so as to cause hair removal and/or follicular devitalization;
wherein the handpiece, the detector, the delivery element and the controller are operably coupled.

2. The apparatus of claim 1 wherein the handpiece is operably coupled to the delivery element, and wherein the delivery element and the detector are operably coupled to the controller.

3. The apparatus of claim 1 wherein the detector detects an effect of a diagnostic form of energy on the portion of skin.

4. The apparatus of claim 1 wherein the detector detects absorption.

5. The apparatus of claim 1 wherein the detector detects color.

6. The apparatus of claim 1 wherein the detector detects an opening of a follicle in the portion of skin.

7. The apparatus of claim 1 wherein the hair removal/follicular devitalization apparatus also comprises a diagnostic energy source that produces diagnostic energy.

8. The apparatus of claim 7 wherein the diagnostic energy is optical energy.

9. The apparatus of claim 7 wherein the diagnostic energy is applied to at an angle substantially perpendicular to a plane of a skin surface.

10. The apparatus of claim 7 wherein the diagnostic energy is applied at an angle of between about 15 degrees and about 85 degrees to a plane of a skin surface.

11. The apparatus of claim 7 wherein the diagnostic energy is applied substantially parallel to a plane of a skin surface.

12. The apparatus of claim 1 wherein the hair removal/follicular devitalization apparatus also comprises a treatment energy source.

13. The apparatus of claim 1 wherein the treatment energy is optical energy.

14. The apparatus of claim 1 wherein the treatment energy is produced by a laser.

15. The apparatus of claim 1 wherein the treatment energy has a wavelength that is selectively absorbed by water.

16. The apparatus of claim 1 wherein the treatment energy has a wavelength that is selectively absorbed by a lipid.

17. The apparatus of claim 1 wherein the treatment energy is sufficient to cause coagulation of a tissue of and/or adjacent to a hair follicle; to cause necrosis of a stem cell within and/or surrounding the bulge region of a hair; to cause necrosis of a cell within and/or surrounding the bulb region of a hair; to coagulate a blood vessel within and/or surrounding the bulb region and/or papilla region of a hair; to cause singeing, charring and/or vaporization of a hair; and combinations thereof.

18. The apparatus of claim 1 wherein the treatment energy has a ring-shaped beam and the beam is centered over the position of the hair and/or follicle.

19. The apparatus of claim 1 wherein the treatment energy is applied at an angle substantially normal to a plane of a skin surface.

20. The apparatus of claim 1 wherein the treatment energy is applied at an angle between about 15 degrees and about 85 degrees to a plane of a skin surface.

21. The apparatus of claim 1 wherein the apparatus further comprises an analyzer operably coupled to the handpiece, the detector, the delivery element and/or the controller.

22. The apparatus of claim 21 wherein the analyzer analyzes the feedback from the detector and calculates at least one target location.

23. The apparatus of claim 21 wherein the analyzer analyzes the feedback from the detector and calculates a target location by comparing absorption spectra.

24. The apparatus of claim 21 wherein the analyzer analyzes the feedback from the detector and calculates a target location by comparing color data.

25. The apparatus of claim 21 wherein a treatment depth is calculated by the analyzer based on the angle at which the hair and/or follicle intersects the plane.

26. The apparatus of claim 1 wherein a treatment depth is predetermined based on a region of a body undergoing treatment.

27. The apparatus of claim 1 wherein a treatment depth is predetermined based on an estimate of a depth of a hair bulge.

28. The apparatus of claim 1 wherein a treatment depth is predetermined based on an estimate of a depth of a hair bulb.

29. The apparatus of claim 21 wherein the analyzer calculates a target location using an offset distance.

30. The apparatus of claim 29 wherein the offset distance is calculated by the analyzer based on the angle at which the hair and/or follicle intersects the plane.

31. The apparatus of claim 1 wherein an offset distance is predetermined based on a region of a body undergoing treatment.

32. The apparatus of claim 1 wherein an offset distance is predetermined based on the angle at which the hair and/or follicle intersects the plane.

33. The method of using a hair removal/follicular devitalization apparatus, wherein the method comprises using an apparatus comprising
a handpiece;
a detector that
detects the presence of a hair and/or follicle in a portion of skin,
detects a position of the hair and/or follicle in a plane, and
detects an angle at which the hair and/or follicle intersects the plane;
a delivery element configured to receive treatment energy from a treatment energy source and deliver the treatment energy to a portion of skin; and
a controller that uses feedback from the detector to control the delivery element and/or to control the treatment energy source so as to control the delivery of the treatment energy to the portion of skin via the handpiece in a manner so as to cause hair removal and/or follicular devitalization;
wherein the handpiece, the detector, the delivery element and the controller are operably coupled.

34. The method of claim 33 wherein the method produces coagulation of a tissue of and/or adjacent to the hair and/or follicle; causes necrosis of a stem cell within and/or surrounding a bulge region of a hair; causes necrosis of a cell within and/or surrounding a bulb region of a hair; causes coagulation of a blood vessel within and/or surrounding a bulb region and/or papilla region of a hair; causes singeing, charring and/or vaporization of a hair; and combinations thereof.
